(19) United States
(12) Patent Application Publication
(10) Pub. No.: US 2009/0222035 A1
(43) Pub. Date: Sep. 3, 2009

(54) Intraluminal Mass Collector

(75) Inventor: Jacob Schneiderman, Kiryat-Ono (IL)

Correspondence Address: MARTIN D. MOYNIHAN d/b/a PRTSI, INC., P.O. BOX 16446, ARLINGTON, VA, 22215, US

(73) Assignee: TEL HASHOMER MEDICAL RESEARCH INFRASTRUCTURE AND S, Ramat-Gan (IL)

(21) Appl. No.: 12/225,662

(22) PCT Filed: Mar. 26, 2007
(86) PCT No.: PCT/IL2007/000390

§ 371 Date: Mar. 30, 2009

Related U.S. Application Data
(60) Provisional application No. 60/786,013, filed on Mar. 27, 2006.

(51) Int. Cl. A61M 29/00; A61M 25/00

(52) U.S. Cl. 606/200; 604/264

(57) ABSTRACT

Disclosed is a catheter for disconnecting, collecting and removing an intraluminal mass from a luminal aspect of a blood vessel, comprising an elongate catheter body, an elongate tubular element extending proximally from an end of the proximal catheter body portion and an intraluminal mass collector configured for collecting an intraluminal mass from a blood vessel. An exemplary collector has a body portion connected to the proximal portion of the catheter body and a radially expandable portion extending in a proximal direction from the body portion, the expandable portion having a reduced diameter configuration with a reduced cross sectional size and at least one expanded diameter configuration. The exemplary catheter additionally includes a disconnector configured for disconnecting an intraluminal mass from a luminal aspect of a blood vessel located proximally from the collector.

The present invention relates generally to minimally invasive intravascular devices and, more particularly, to devices used to disconnect, collect, and remove an intraluminal mass from a luminal aspect of a blood vessel.

The present invention is related to U.S. patent application Ser. No. 11/290,450, filed on Dec. 1, 2005 and to U.S. Provisional Patent Application No. 60/726,618, filed on Oct. 17, 2005, both which are incorporated by reference as if fully set forth herein.

### FIELD AND BACKGROUND OF THE INVENTION

Aortic Arch Protruding Thrombus (AAPT) is a unique clinical entity involving a thrombus that emerges off the aortic luminal wall along the proximal aorta; including the ascending segment, arch segment, and proximal descending segment of the aorta. AAPT is associated with life threatening emboli of occluding blood clots that are shed from the AAPT into arteries of the brain, internal organs and extremities.

FIG. 1A is a representation of an aorta 100 connected to a heart 144, showing an AAPT 170 in a proximal aorta 140. AAPT 170 typically projects into a blood vessel lumen 148 from a thin stalk 172 attached to a luminal aspect 152 of proximal aorta 140.

In a study of 22 cases, most AAPT's 170 were located in a distal arch 199. Five were located adjacent to an innominate 130 artery, a left carotid 120 artery or a left subclavian 110 artery. (“Mobile Thromboses of the Aortic Arch Without Aortic Debris”, Theirry Laperche et al, “Circulation” 1997; 96: 288-294)

AAPT 170 comprises a typical thrombus composition, including fibrin, platelets, and blood cells. Due to the blood motion and beating of heart 144, AAPT 170 partially disintegrates, shedding one or more fragments as embolus 180. Embolus 180 may lodge, for example, in a celiac artery 132, a superior mesenteric 124 artery, a renal artery 122, or other organ-related blood vessel, causing tissue necrosis in associated organs, for example the spleen or intestine.

In FIG. 1A, an embolus 182 is shown entering a superior mesenteric 124 artery, thereby blocking circulation to a portion of the upper intestines (not shown), likely causing ischemia and necrosis of a portion of the intestines. Necrosis of a portion of any internal organ is a medical emergency that typically requires open surgery and resection of the necrotic tissue.

AAPT 170 is considered responsible for approximately 3% of all peripheral emboli originating from a central source. AAPT 170 generally occurs in relatively young people that have no history of coronary or peripheral atherosclerosis, but may have high blood pressure, an undiagnosed tendency for arterial thrombosis and/or may be heavy smokers.

The pathogenesis of AAPT 170 has been attributed to rupture of a soft shallow atherosclerotic plaque located in the aortic arch and appears to be related to the exposure of necrotic core components to the blood stream; the core components including tissue factor, PAI-1 and ox-LDL. Formation of emboli from AAPT 170 can be compounded by pre-existing thrombophilia or a transitory pro-thrombotic state.

AAPT 170 is often first diagnosed on an ultrasound image that is made following a serious embolic incident, for example necrosis of a portion of the intestine or other internal organs. Systemic therapy with anticoagulants has not proven beneficial in preventing further emboli after the initial embolic episode.

To ensure that AAPT 170 does not cause further necrosis of other organ tissue, within a short period following removal of the necrotic organ tissue, the patient must be subjected to an open chest surgery to remove AAPT 170. Open chest surgery is a major cardiovascular surgical procedure that includes cardiopulmonary bypass, deep hypothermia and arrest of the systemic circulation, all associated with high morbidity and mortality.

U.S. patent application Ser. No. 11/290,450, filed 1 Dec. 2005, of the inventor, teaches a method for disconnecting an AAPT using, inter alia, a large balloon catheter. The catheter is used to disconnect AAPT 170 from luminal aspect 152 of proximal aorta 140 so that AAPT 170 passes in a direction 118 through lumen 148 along with the blood flowing through lumen 148. AAPT 170 is then collected downstream, typically in a common iliac artery 194 branch, for example a right 134 or a left 135 femoral artery.

A very real concern of the catheter procedure is that AAPT 170 may break up during or following detachment from stalk 172 and lodge in a critical branch of the aorta, causing, for example, organ necrosis. This is of particular concern when the procedure is performed by an inexperienced surgeon or when AAPT 170 is located in an irregularly shaped aorta 100, making disconnection difficult.

Small vessel embolic debris collection devices are known, but would not be effective in disconnecting, collecting and removing AAPT 170. U.S. Pat. No. 4,873,978 to Ginsburg, for example, teaches a collection device, without a means of disconnecting AAPT 170, which must be retracted into a small diameter catheter, likely causing a disastrous breakup of AAPT 170.

U.S. patent application Ser. No. 10/854,920, published as US 2005/0277976 to Galdonik et al., teaches a three-dimensional matrix designed to filter and route small amounts of embolic debris into a tiny catheter opening.

If the Galdonik device were used for AAPT disconnection, collection and removal, the filtering matrix would likely cause breakup of AAPT 170. Since the filtering matrix does not fully span the lumen, chunks of AAPT 170 would easily bypass the filter causing the above-noted disastrous consequences. Additionally, the Galdonik filter matrix is not collapsible so enlarging the filtering matrix would require open chest surgery and introduction directly into the aorta, the very procedure that must be avoided in dealing with AAPT 170.

In spite of the need for a minimally invasive device for disconnecting, collecting and removing AAPT 170, there are presently no such devices available. The lack of an appropriate device allowing rapid disconnection, collection and removal of AAPT 170 means that by default, open chest surgery, with its high associated risks of morbidity, remains the procedure of choice.

### SUMMARY OF THE INVENTION

The present invention successfully addresses at least some of the shortcomings of the prior art by providing a device configured for the capture of an AAPT.

According to the teachings of the present invention, there is provided a catheter for disconnecting, collecting and removing an intraluminal mass from a luminal aspect of a blood vessel, comprising an elongate catheter body having a proximal portion and a distal portion, an elongate tubular element extending proximally from an end of the proximal catheter body portion, and an intraluminal mass collector configured for collecting an intraluminal mass from a blood vessel.

In a embodiments, the catheter has a body portion connected to the proximal portion of the catheter body and a radially expandable portion extending in a proximal direction from the body portion and surrounding at least a portion of the elongate tubular element, the expandable portion having a reduced diameter configuration with a reduced cross sectional size and at least one expanded diameter configuration, each expanded diameter configuration having a respective expanded cross sectional size. Additionally, in embodiments the catheter comprises a disconnector configured for disconnecting an intraluminal mass from a luminal aspect of a blood vessel, the disconnector connected to a proximal portion of the elongate tubular element at a distance from the collector.

In embodiments, the collector is configured to strain an intraluminal mass from the blood when in the expanded diameter configuration.

In embodiments, the catheter includes a catheter sleeve slidably associated with at least a portion of the catheter body and configured to surround at least a portion of the collector in the reduced diameter configuration, prior to deployment.

In embodiments, the collector includes at least two substantially resilient rays extending from the collector body portion in a proximal direction towards an end of the radially expandable portion.

In embodiments, the collector has a diameter that is configured to span the large diameter of the aorta, typically between three and five centimeters and gently conforms to the often highly irregular aortic shape.

In embodiments, the body portion of the intraluminal mass collector comprises a ring-shaped component connected to the catheter body.

In embodiments, the at least two rays are attached to the ring using a process selected from the group including welding, adhesion, gluing and riveting.

In embodiments, proximal portions of each of the at least two rays are configured to resiliently flex outward to form at least one expanded cross sectional diameter; the extent of the outwards flexing is configured to be limited by the walls of a vessel in which the collector is deployed. In embodiments, the each of the at least two rays is configured to resiliently flex outward to form the at least one expanded cross sectional diameter.

In embodiments, the collector is configured to effectively collect a large AAPT and, accordingly, includes a sheet material operatively associated with the at least two rays, the material preferably forming a substantially conical shape pointing in a distal direction when the at least two rays are in an expanded diameter configuration. In embodiments, each of the rays has an internal and an external aspect and the material is attached to at least one of the internal aspects and the external aspects.

In embodiments, the material is attached to at least one of the at least two rays using a process selected from the group of sewing, adhesion, gluing, suturing, riveting and welding.

The collector is preferably configured to allow blood flow through the lumen while in the expanded state. In embodiments, the sheet material is selected from the group consisting of meshes and nets.

In embodiments, the material extends proximally beyond at least one of the at least two rays.

In embodiments, the material is from the group including a synthetic biostable polymer, a natural polymer, and an inorganic material.

In embodiments, the natural polymer is selected from the group consisting of cotton, linen and silk.

In embodiments, the catheter further includes at least one elongate flexible biasing element, having a first end attached to a first portion of the catheter sleeve, a second end attached to a second portion of the catheter sleeve, and a body between the first and second ends, the body being operatively associated with a proximal portion of each of the at least two rays.

In embodiments, the at least one flexible biasing element is configured to bias at least one of the at least two rays from an expanded diameter configuration to a smaller diameter configuration.

In embodiments, the biasing element is selected from the group consisting of wires, strings, threads, springs, ribbons, filaments, cables, yarn, and ropes.

In embodiments, a passage is operatively associated with a proximal portion of the at least one ray through which the body of the at least one elongate flexible biasing element passes.

In embodiments, the passage is formed from at least one of a bending of the proximal portion of the ray, and a shaped component attached to the proximal portion of the ray.

In embodiments, the catheter body includes a continuous aspiration channel from the distal portion and emerging into the collector body portion.

In embodiments, the catheter further includes a collector ray converger comprising a curved wall that slidingly substantially encircles a portion of the distal portion of the catheter body.

In embodiments, the collector ray converger is additionally configured to encircle at least a portion of at least one of the collector, and the catheter sleeve.

In embodiments, the collector ray converger is configured to provide a radially inward force on at least one of the at least two rays.

In embodiments, the collector ray converger is configured to reduce an intraluminal mass diameter, when the mass has been collected in the collector.

In embodiments, the catheter further comprises a balloon used in disconnecting an AAPT from a luminal aspect of a blood vessel. Preferably but not necessarily, the disconnector balloon is configured to inflate by introduction of a fluid through an inflation channel running through the catheter body and the elongate tubular element.

In embodiments, the balloon comprises a material from the group including rubber, silicon rubber, latex rubber, polyethylene, polyethylene terephthalate, and polyvinyl chloride.

In embodiments, the catheter body includes a substantially coaxial guide wire channel.

According to the teachings of the present invention, there is also provided a method for collecting emboli shed into circulation within the vascular system, the method comprising expanding an expandable lumen blocker on a first side of shed emboli within the vascular system, opening a collector on a second side of the emboli, moving the lumen blocker to contact the shed emboli so as to move shed emboli toward the collector, collecting the emboli within the collector, and closing the collector, thereby containing the shed emboli within the collector.

In embodiments of the method, the first side is proximal and the second side is distal. Alternatively, the first side is distal and the second side is proximal.

In embodiments, the method further includes treating a portion of a stenosed region using dilation.

In embodiments, the method further includes treating a portion of a stenosed region using laser ablation.

In embodiments, the method further includes treating a portion of a stenosed region by atherectomy.

In embodiments, the method further includes aspirating the shed emboli from the collector.

### BRIEF DESCRIPTION OF THE DRAWINGS

The invention for safely disconnection of an AAPT using a minimally invasive vascular surgical technique is described by way of example with reference to the accompanying drawings. With specific reference now to the drawings in detail, it is stressed that the particulars shown are by way of example and for purposes of illustrative discussion of the preferred method of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the methods of the invention may be embodied in practice.

FIG. 1A (prior art) is a representation of an in situ AAPT, in accordance with an embodiment of the present invention;

FIGS. 1B, 2A-2C and 3A-3C are representations of portions of a catheter for collecting an in situ AAPT, in accordance with an embodiment of the present invention;

FIG. 4A is a representation of a Transoesophageal Echocardiograph (TEE) setup in accordance with an embodiment of the present invention;

FIGS. 4B, 5, 6, 7, 8 and 9 demonstrate a minimally invasive technique using the a catheter based collector tool shown in FIG. 3B, in accordance with an embodiment of the present invention;

FIGS. 10, 11 and 12 are cross sectional representations of the apparatus shown in FIG. 5, in accordance with an embodiment of the present invention; and

FIG. 13 is an alternative embodiment of the collector shown in FIG. 5, in accordance with an embodiment of the present invention.

### DESCRIPTION OF THE PREFERRED EMBODIMENTS

In broad terms, the present invention relates to an apparatus for disconnecting, collecting and removing an AAPT using a minimally invasive vascular surgical technique.

The principles and uses of the teachings of the present invention may be better understood with reference to the accompanying description, figures and examples. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of biology, engineering, material sciences, medicine and physics. Such techniques are thoroughly explained in the literature.

Unless otherwise defined, all technical and scientific terms used herein have the same meaning as commonly understood by one of ordinary skill in the art to which the invention belongs. In addition, the descriptions, materials, methods, and examples are illustrative only and not intended to be limiting. Methods and materials similar or equivalent to those described herein can be used in the practice or testing of the present invention.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, “a” or “an” mean “at least one” or “one or more”. The use of the phrase “one or more” herein does not alter this intended meaning of “a” or “an”.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

As used herein, the terms proximal and proximally refer to positions and movement respectively toward the heart. As used herein, the terms distal and distally refer to positions and movement respectively away from the heart.

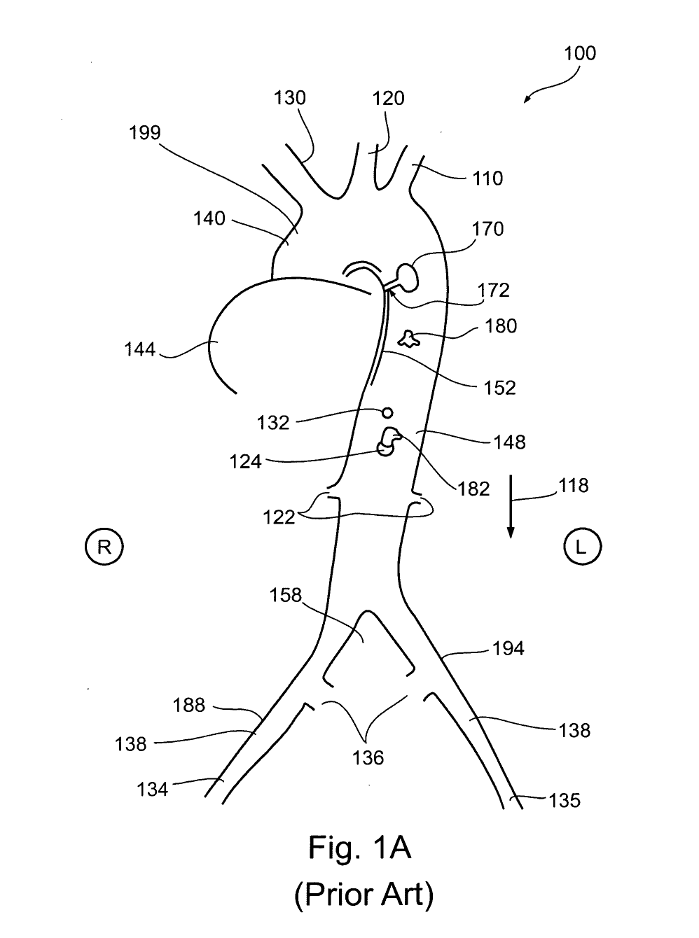

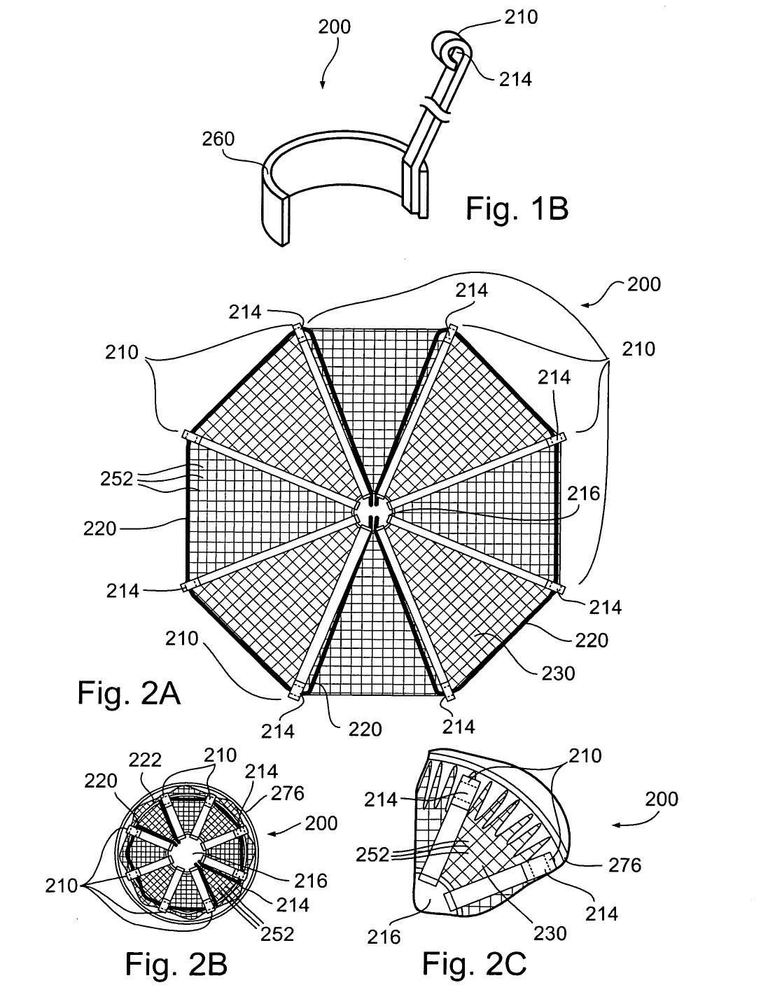

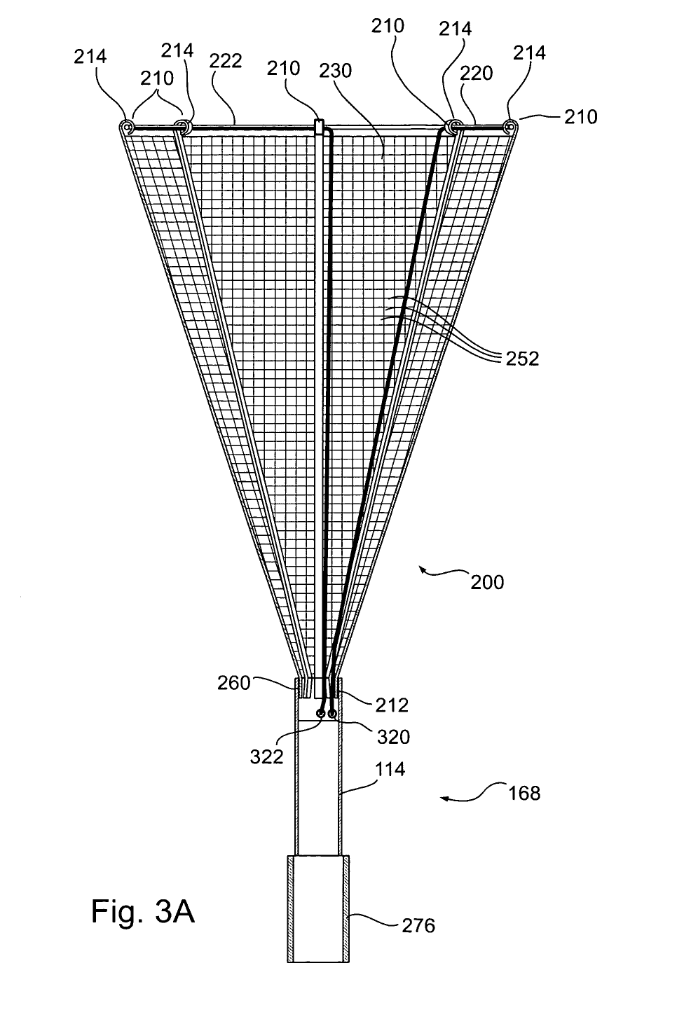

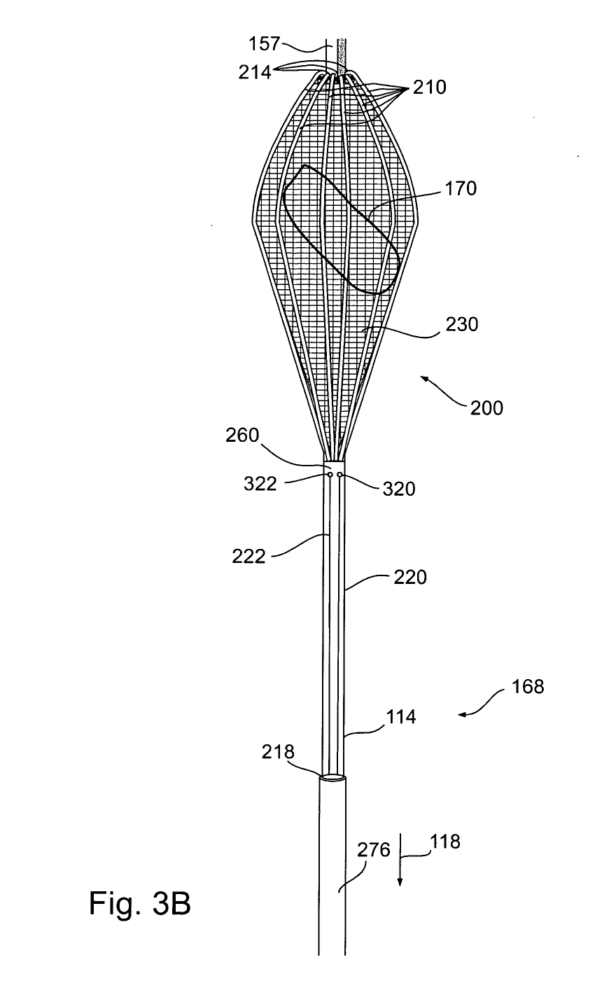

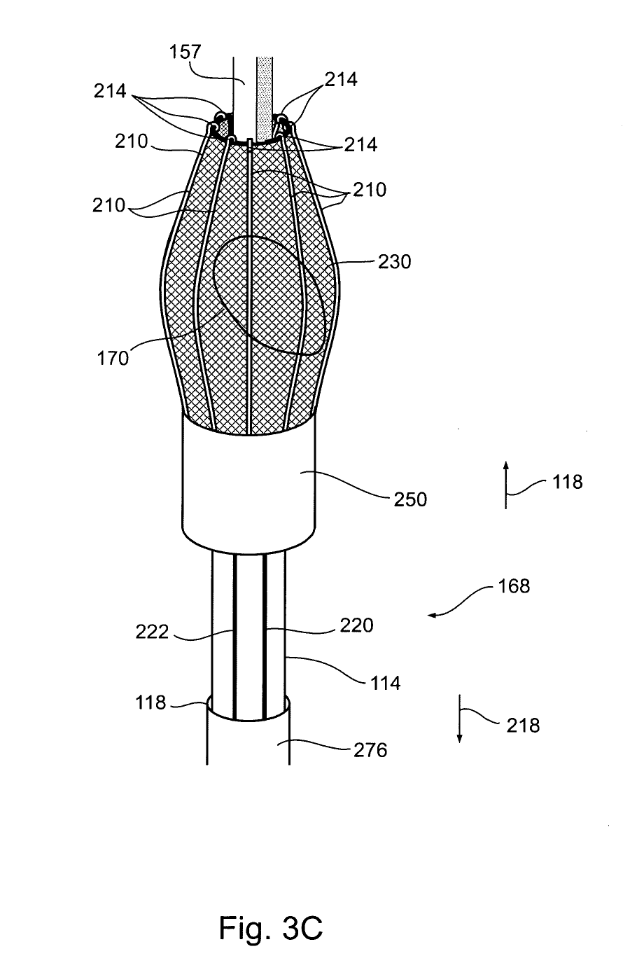

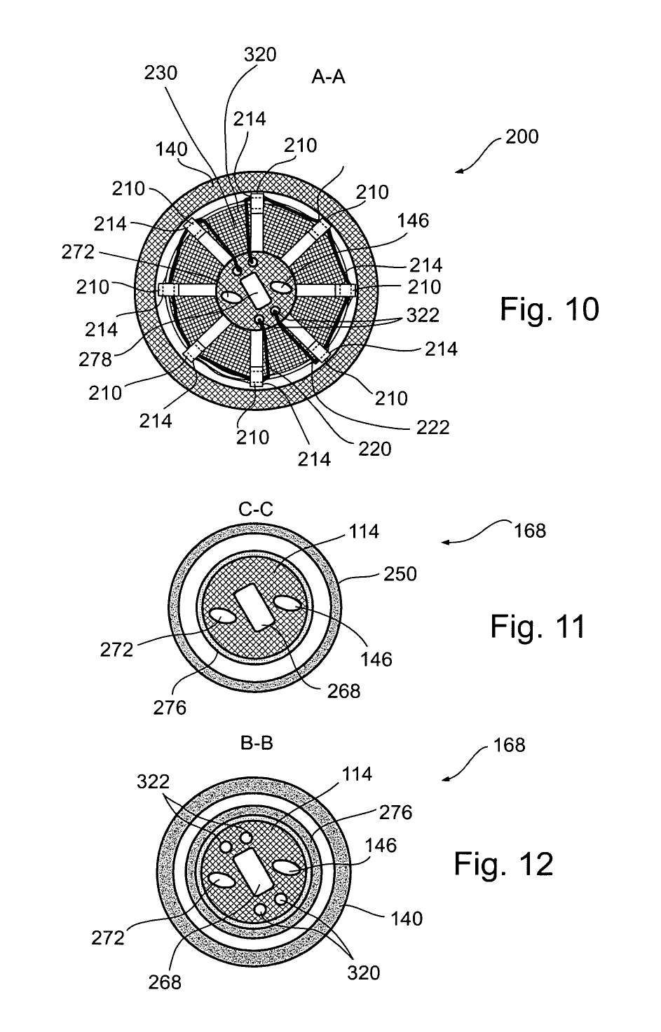

FIGS. 1B, 2A-2C and 3A-3C are representations of portions of an AAPT collector 200 of the present invention. FIG. 3A is a cut-away of a catheter 168, showing a cross-section of catheter sheath 176, catheter body 114 and collector 200, in an expanded state. Collector 200 comprises multiple rays 210 projecting upward and radially outward from a base 260 at the proximal end of catheter body 114. Spanning rays 210 is a mesh 230. During collection of an AAPT, collector 200 preferably disrupts blood flow as little as possible and to this end, mesh 230 includes relatively large openings 252, for example 1×1 (1 mm²) millimeter each, that allow substantial flow of blood there through while collector 200 is expanded. An aerial view of collector 200 in an expanded state, in a cross section of proximal aorta 140, is seen in FIG. 10.

As seen in FIG. 1B, each ray 210, attached to base 260, includes an eye 214 comprising a guide passage for strings 220 and 222. As seen in FIG. 2A, an aerial view of collector 200, a first string 220 passes through four eyes 214 and a second string 222 passes through four eyes 214. The two ends of each of strings 220 and 222 pass through a central collector opening 216. FIGS. 11 and 12 are cross sections of upper and lower catheter body 114 portions respectively. String conduits 322 and 320 demonstrate an exemplary embodiment of the upper portion of catheter body 114. Both upper and lower portions of catheter body 114 include a guide wire channel 268, a saline channel 146 and an optional aspirator channel 272.

As seen in FIG. 3B, strings 220 and 222 pass from the internal to external portion of base 260 through string conduits 320 and 322 respectively, and attach to an edge 218 of a catheter sheath 276. By pulling sheath 276 in direction 118, strings 220 and 222 cause rays 210 to bend radially inward toward a guide wire 157 and thereby trap an AAPT 170.

As seen in FIG. 3C, to facilitate removal of AAPT 170 from a smaller diameter branch artery, as noted above, a ray converger 350 is included with collector 200 to reduce the diameter of rays 210 as described further on.

As shown in FIGS. 10 through 12, catheter body 114 optionally includes aspirator channel 272 that may be used to aid in reducing bulk by aspirating all or a portion of AAPT 170.

Aspirated AAPT 170, having a smaller diameter and/or less bulk, is removed from the branch vessel more easily than with ray converger 230 alone. Due to the closed shape of collector 200 and direction 118 of blood flow, AAPT 170 remains contained within collector 200.

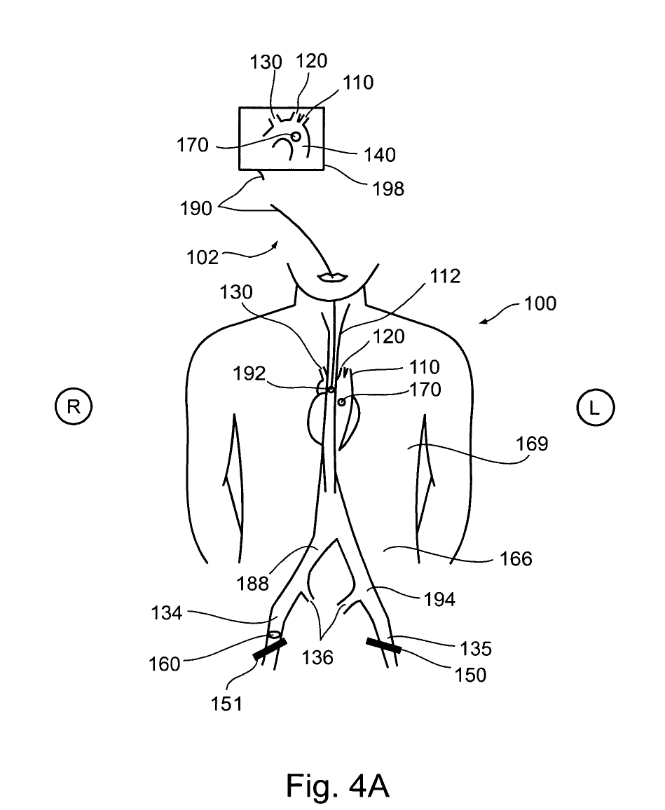

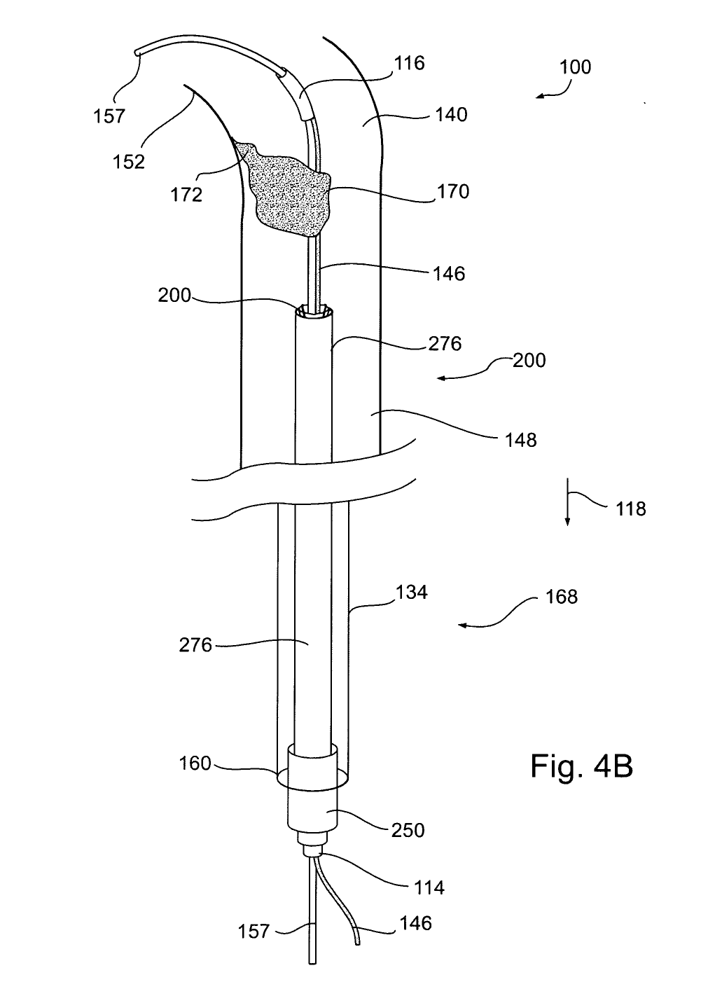

FIG. 4A is a representation of a Transoesophageal Echocardiograph 102 (TEE) setup used for diagnosing and removal of AAPT 170. TEE 102 includes an ultrasound echo probe 192 having an ultrasound cable 190 that is passed through an esophagus 112 in a human 166. In the position shown, probe 192 demonstrates the position of AAPT 170 on a monitor 198.

While TEE 102 is shown in exemplary embodiments, other methods and/or monitoring systems and/or imaging modalities may be utilized, inter alia, intraoperative CT, MRI and nuclear imaging.

Prior to beginning the AAPT removal procedure in accordance with the teachings of the present invention, the surgeon typically places a clamp 150 on a left femoral artery 135 and a second, more distal, clamp 151 on a right femoral artery 134, thereby preventing distal embolization during the procedure. An incision 160 is made proximal to clamp 151, allowing access to right femoral artery 134 and retrograde maneuvering of guide wire 157 and collector 200 (FIG. 3B).

In an alternate exemplary embodiment, noted above, internal iliac arteries 136, (branching off left 194 and common right 188 iliac arteries) are clamped with clamps 150 and 151 respectively and an incision (not shown) is made.

As seen in FIG. 4B, guide wire 157 is central to an inflatable balloon 116 that is used in disconnecting AAPT 170 from luminal aspect 152. Disconnector balloon 116 is connected to saline channel 146 passing along guide wire 157 and out the base of catheter 114.

FIG. 2B shows an aerial view of collector 200 in the collapsed state contained within catheter sheath 276. FIG. 2C shows a detail of mesh 230 folded between rays 210 with collector 200 in the collapsed state.

In the collapsed state, collector 200 is passed through incision 160, retrograde to a direction of blood flow 118 until balloon 116 is proximal to AAPT 170.

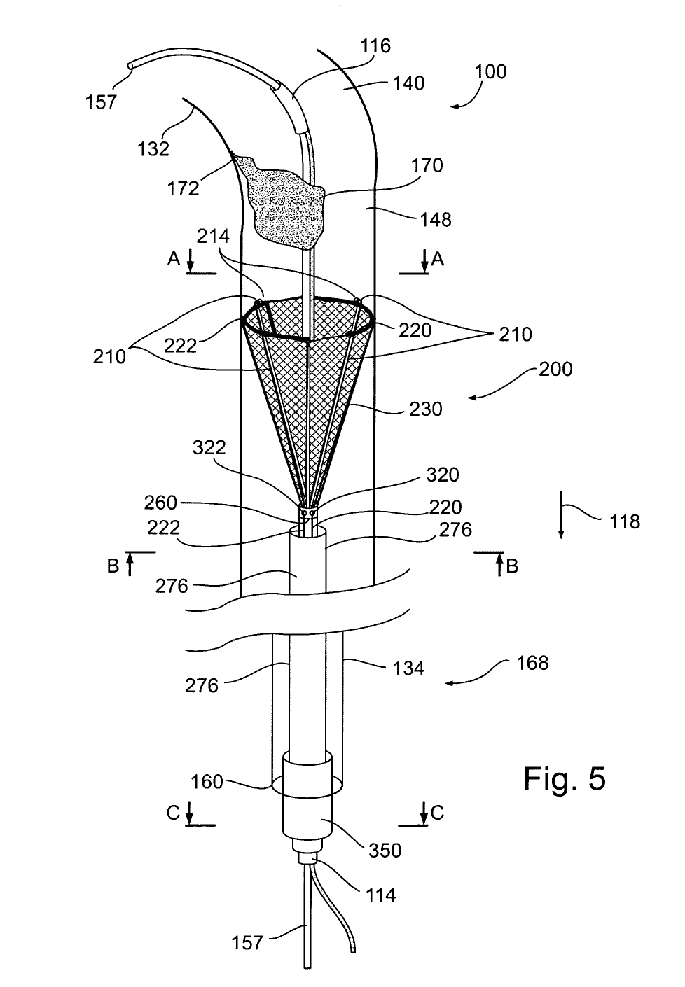

As seen in FIG. 5, with balloon 116 proximal to AAPT 170, sheath 276 is pulled in a direction 118 with respect to catheter body 114 so that rays 210 gently radially expand against a luminal aspect of proximal aorta 140.

The resilient nature of each ray 210 allows gentle pressure against respective luminal aspects 152. Additionally, each ray 210 seeks its own outward radial distance from guide wire 157 so that collector 200 easily conforms to aortas 100 having irregular shapes without causing damage to luminal aspect 152.

As noted above, rays 210 comprise a resilient material from the group including titanium, stainless steel, nitinol, shape memory metals, synthetic biostable polymer, a natural polymer, and an inorganic material. The many variations of, for example, polymers being well known to those familiar with the art.

Additionally, while eight rays 210 are shown, embodiments of collector 200 include as few as about six rays or as many as about 12 rays 120.

In an exemplary embodiment, mesh 230 includes openings having an area of at least about 0.25 mm², or no more than about 1.5 mm². Further, mesh 230 optionally extends proximally beyond rays 210 to aid in capturing AAPT 170 when collector is collapsed, as well as to provide a gentle interface between rays 210 and luminal aspect 152.

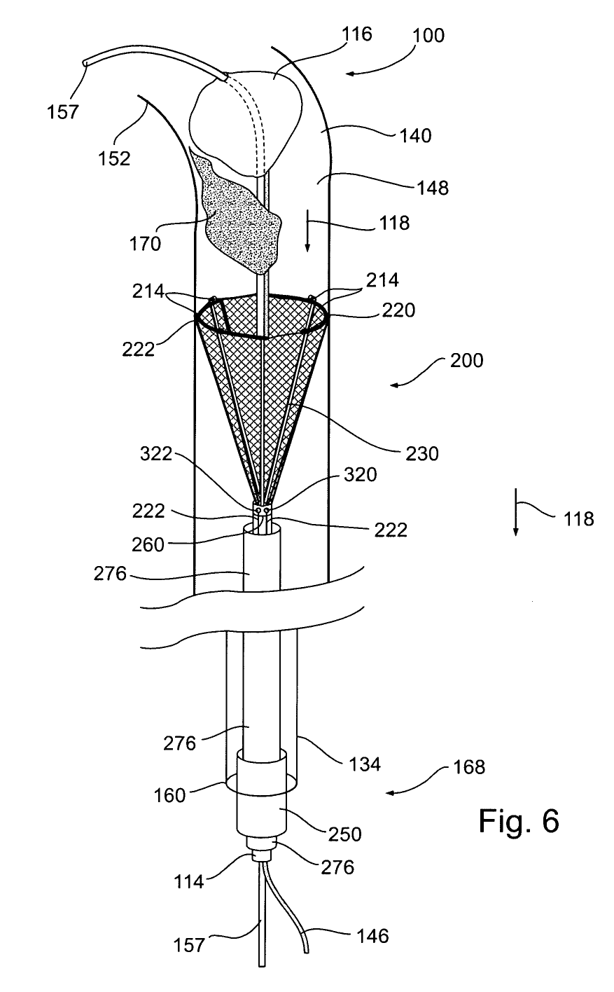

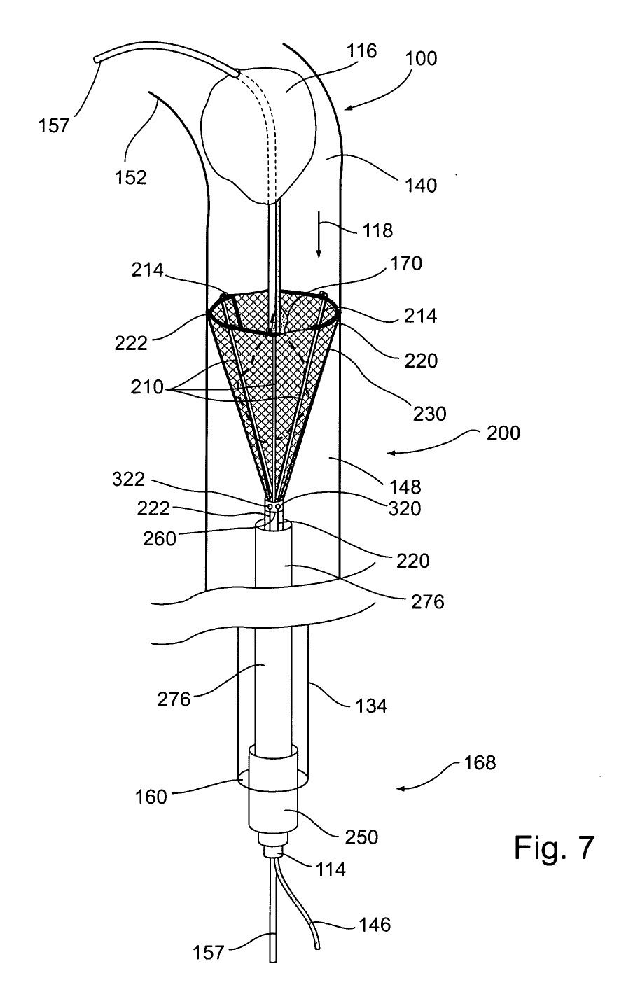

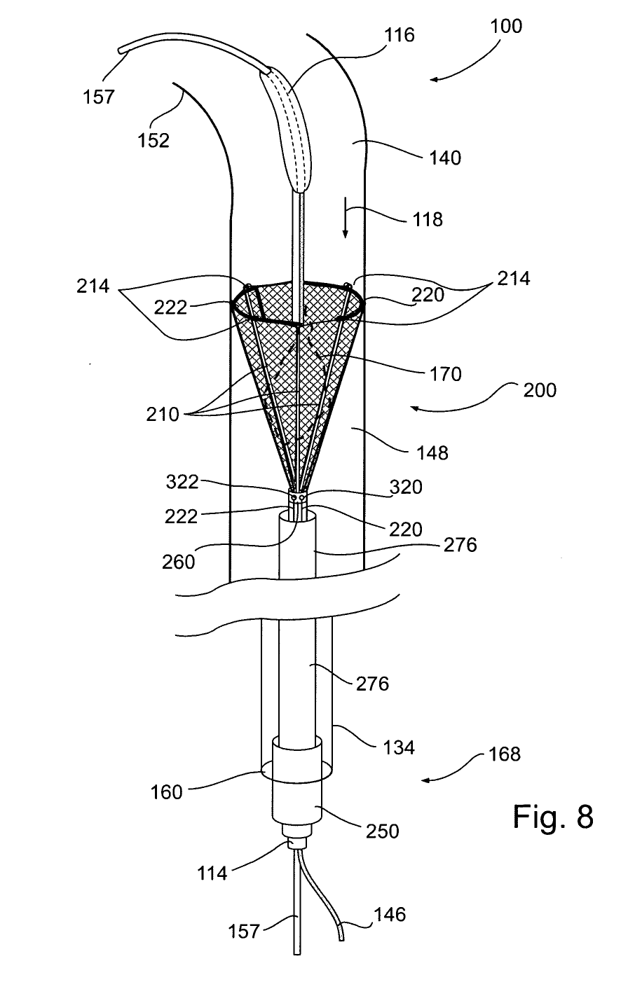

As seen in FIG. 6, balloon 116 has been inflated, for example with pressurized sterile saline through channel 146. After inflation balloon 116 is gently pulled distally (direction 118) along guide wire 157 to contact AAPT 170. As a result of contact between balloon 116 and AAPT 170, AAPT 170 is disconnected from stalk 172.

In an exemplary embodiment, disconnector balloon 116 has a large diameter to expand sufficiently to fill the large diameter of the lumen of proximal aorta 140 for example, a maximum inflation radius of at least about 2 centimeters, or no more than about 15 centimeters.

Additionally, balloon 116 includes flexible walls, for example comprising latex or the like, so as to gently conform to the aortic walls to preclude damage thereto. In some embodiments, disconnector balloon 116 has a wall thickness of at least about 0.2 millimeters up to no more than about 0.5 millimeters. The many materials and measurements that are optionally used in the manufacture of balloon 116, are well known to those familiar with the art.

Balloon 116 typically expands to at least about 3.0 centimeters in diameter. In an exemplary embodiment, balloon 116 is in an inflated state or a partially inflated state for no more than 20 seconds, no more than 15 seconds and even no more than about 10 seconds. Such a short time span lowers the chance of hemodynamic instability caused by a significant period of blood flow stoppage.

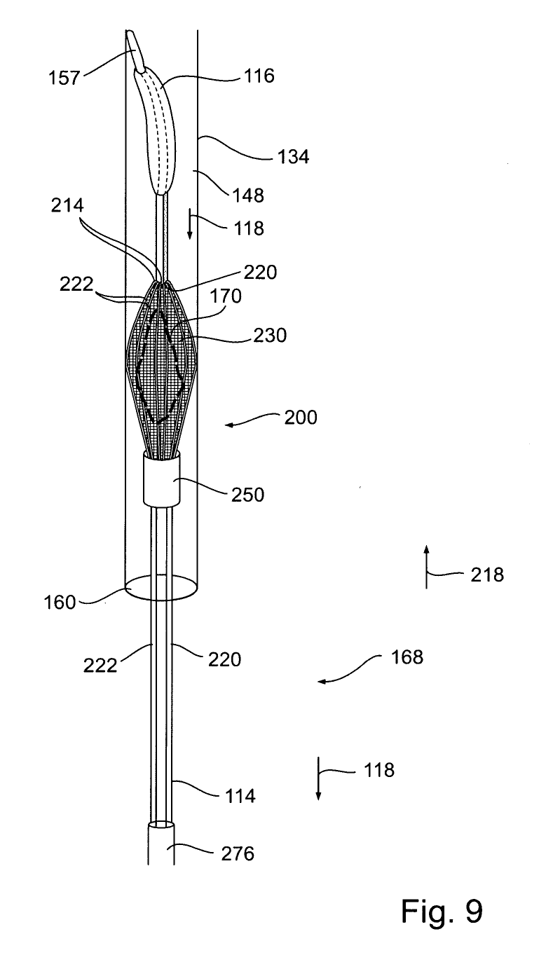

In embodiments of the invention, once released, AAPT 170 floats as one intact mass into expanded collector 200. As seen in FIG. 9, and noted above, pulling sheath 176 in a direction 118 puts tension on strings 220 and 222, thereby bending rays 210 and trapping AAPT 170 within collector 200.

In exemplary embodiments, catheter 168 (including catheter body 114, catheter sheath 276, collector 200, guide wire 157 and balloon 116) is pulled outwards in direction 118 until proximal to right femoral artery 134.

Ray converger 350 is then moved in direction 218 within femoral artery 134 while stabilizing the position of catheter 168 with Ray converger 350 is pressed distally against rays 210, thereby causing rays 210 to bend and reshape AATP 170 as described above.

With rays 210 bent, AAPT 170 is forced to form a longer shape with a narrow diameter, thereby more easily fitting through artery 134 and incision 160. Those familiar with the art know that artery 134 has the ability to expand to a larger diameter, for example about 6.5 millimeters, thereby additionally facilitating removal of collector 200 from incision 160.

Removal of balloon 116 and guide wire 157 follows removal of AAPT 170, and incision 160 is closed, for example with a suture or surgical clips in the usual way.

In embodiments of the invention, drugs are administered post-operatively to prevent recurrence of an AAPT 170.

Typically, assuming the patient has prothrombotic tendencies, anticoagulant therapy will be administered for life.

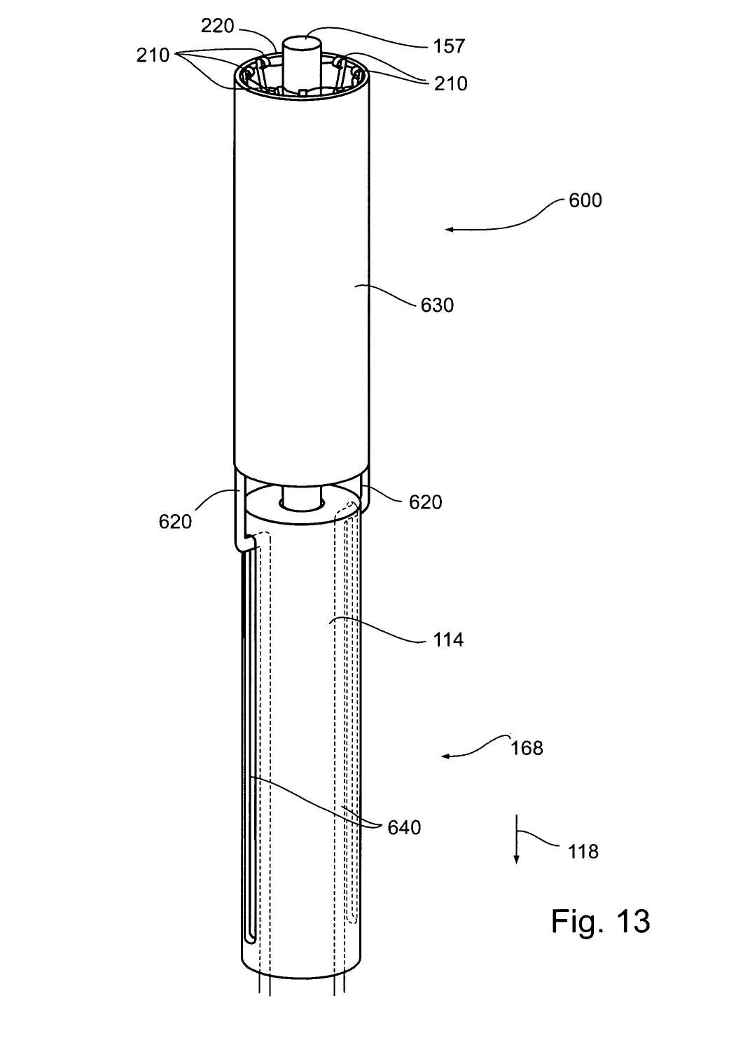

An alternative collector embodiment 600, seen in FIG. 13, has a short, retractable, collector sheath 630 that maintains collector rays 210 in a collapsed state during insertion. Collector sheath movement is controlled by legs 620 passing through slots 640 in catheter body 114 and internal through the length of catheter body 114. By pulling legs 620 in direction 118 while catheter body 114 is stabilized, sheath 630 is removed from rays 210, allowing radial expansion of collector 200.

The closure of collector rays 210 uses strings 220 and 222, in the same manner as noted above. Additionally, rays 210 of collector 600, upon reaching a narrower artery, for example right femoral artery (FIG. 4A) will be bend radially inward using, for example, converger 250 in the manner shown in FIG. 9.

### Materials and Specifications

Attention will be now directed at typical materials and dimensions of a device of the present invention.

Generally, collector 200 is configured to span the large diameter of proximal aorta 140, typically between three and five centimeters and to gently conform to the often highly irregular aortic shape. Thus, there are typically at least about 6, 8 even 10 rays 210. Typically, there are no more than about 16 or 12 rays 210.

Typically, at least one of rays 210 has a substantially circular cross section having a diameter of at least about 0.1 millimeters, about 0.2 or even about 0.3 millimeters. Typically, at least one of rays 210 has a substantially circular cross section having a diameter of no more than about 0.6 millimeters, about 0.4 millimeters or about 0.5 millimeters.

In embodiments, at least one of rays 210 has a cross section having greater and lesser measurements, for example, oval or rectangular. Typically the greater measurement is at least about 0.1 millimeters, about 0.2 millimeters, about 0.3 millimeters, or even about 0.4 millimeters. Typically, the greater measurement is no more than about 0.6 millimeters, about 0.5 millimeters, or even about 0.4 millimeters.

Typically the lesser cross sectional measurement is at least about 0.1 millimeters, about 0.2 millimeters, and even at least about 0.3 millimeters. Typically, the lesser cross sectional measurement is no more than about 0.6 millimeters, about 0.5 millimeters, or even about 0.4 millimeters.

In embodiments, rays 210 are attached to ring portion 260 of the catheter body using a process selected from the group including welding, adhesion, gluing and riveting.

Typically, the proximal portions of each of rays 210 are configured to resiliently flex outward to form a maximally expanded cross section of at least about 3 centimeters, about 4 centimeters, or even at least about 5 centimeters. Generally, the expanded cross sectional diameter is no more than about 10 centimeters about 7 centimeters, about 8 centimeters, or even no more about 9 centimeters. The maximum extent of expansion is generally limited by material 230.

Collector 200 is configured to effectively collect an AAPT and, accordingly, includes a sheet material 230 operatively associated with rays 210. Typically, material 230 is attached to at least one of the internal aspects and the external aspects of rays 210. Typically, material 230 is attached to at least one of rays 210 using a process selected from the group of sewing, adhesion, gluing, suturing, riveting and welding.

Collector 200 is preferably configured to allow blood flow through lumen 148 while in the expanded state. In embodiments, sheet material 230 is selected from the group consisting of meshes and nets.

To allow minimal interruption of blood flow, material 230 typically includes relatively large openings 252. Typically openings 252 have an area of at least about 0.25 mm², about 0.5 mm², about 1.0 mm², about 1.5 mm², about 2.25 mm², or even about 4.0 mm². In embodiments, openings 252 have an area of no more than about 4.0 mm², about 2.25 mm² mm², about 1.5 mm², or even about 1.0.

In embodiments, material 230 extends proximally beyond at least one of rays 210 by at least about 1.0 millimeter, about 2.0 millimeters, about 3.0 millimeters, or even by at least about 4.0 millimeters. Typically, material 230 extends proximally beyond at least one of rays 210 by no more than about 2.0 millimeters, about 3.0 millimeters, or about 4.0 millimeters.

In embodiments, catheter 168 further includes at least one elongate flexible biasing element, for example strings 220 and 222, configured to bias at least one of rays 210 inwardly causing collector 200 to close from an expanded diameter configuration to a smaller diameter configuration. Typically, biasing element 220 is selected from the group consisting of wires, strings, threads, springs, ribbons, filaments, cables, yarn, and ropes.

Typically, a flexible biasing element has a diameter of at least 0.2 millimeters, about 0.3 millimeters, about 0.5 or about 0.6 millimeters. Typically, a flexible biasing element has a diameter of no more than about 0.8 millimeters, about 0.3 millimeters, about 0.5 about 0.6 millimeters, or about 0.7 millimeters.

In embodiments, passage 214 is operatively associated with at least one ray 210 through which the body of the at least one elongate flexible biasing element 220, 222 passes.

In embodiments, passage 214 is formed from at least one of a bending of the proximal portion of the ray, and a shaped component attached to the proximal portion of the ray.

In embodiments, catheter 168 further includes a collector ray converger 350 configured to encircle at least a portion of at least one of collector 200, and the catheter sleeve 276.

In embodiments, collector ray converger 350 has a length of at least about 3 centimeters, about 4 centimeters, about 5 centimeters, or about 6 centimeters. In embodiments, collector ray converger 350 has a length of no more than about 7 centimeters, about 6 centimeters, about 5 centimeters, or even about 4 centimeters.

In embodiments, collector ray converger 350 wall has a thickness of at least about 0.3 millimeters, about 0.4 millimeters, or at least about 0.5 millimeters. In embodiments, collector ray converger 350 wall has a thickness of no more than about 0.6 millimeters, about 0.4 millimeters, or even about 0.5 millimeters.

In embodiments, catheter 168 further comprises balloon 116 used in disconnecting AAPT 170 from a luminal aspect 148, comprising a material from the group including rubber, silicon rubber, latex rubber, polyethylene, polyethylene terephthalate, and polyvinyl chloride.

In embodiments, balloon 116 has a maximum inflation radius of at least about 2 centimeters, at least about 3 centimeters, about 4 centimeters, about 5 centimeters, about 6 centimeters, or about 7 centimeters. In embodiments, the expanded cross sectional diameter is no more than about 15 centimeters, about 10 centimeters, or about 12 centimeters.

In embodiments, the inflatable balloon 116 has a wall thickness of at least about 0.2 millimeters, about 0.3 millimeters, or about 0.4 millimeters. In embodiments, inflatable balloon 116 has a wall thickness of no more than about 0.5 millimeters, about 0.4 millimeters, or even about 0.3 millimeters.

In embodiments, the distance from disconnector balloon 116 to the proximal end of collector 200 in the reduced diameter configuration is at least about 5 centimeters, about 6 centimeters, about 7 centimeters, about 8 centimeters, about 9 centimeters, about 10 centimeters, or about 11 centimeters. In embodiments, the distance from disconnector 116 to the proximal end of collector 200 in the reduced diameter configuration is no more than about 12 centimeters, about 11 centimeters, about 10 centimeters, about 9 centimeters, about 8 centimeters, about 7 centimeters or even about 6 centimeters, or.

In embodiments, catheter body 114 includes a substantially circular coaxial guide wire channel 268 having a substantially circular cross section with a typical diameter of at least about 0.4 millimeters, about 0.8 millimeters, or about 1.2 millimeters. In embodiments, guide wire channel 268 has a substantially circular cross section with a diameter of no more than about 1.5 millimeters, about 1.2 millimeters, or about 0.8 millimeters.

In a further exemplary embodiment, guide wire channel 268 includes greater and lesser cross sections (e.g., is oval or rectangular). Typically, the greater cross section is at least about 0.1 millimeters, about 0.2 millimeters, or about 0.3 millimeters. In embodiments, the greater cross section is no more than about 0.4 millimeters, about 0.2 millimeters, or about 0.3 millimeters. Typically, the lesser cross section is at least about 0.1 millimeters about 0.2 millimeters, or about 0.3 millimeters. In embodiments, the lesser cross section is no more than about 0.4 millimeters, about 0.2 millimeters, or about 0.3 millimeters.

Typically, catheter body 114 has an outside diameter of at least about 3.0 millimeters, about 3.5 millimeters, about 4.5 millimeters, about 5.0 millimeters, or about 5.5 millimeters. In embodiments, catheter body 114 has an outside diameter of no more than about 5.5 millimeters, about 5.0 millimeters, about 4.5 millimeters, or about 4.0 millimeters.

Typically, catheter body 114 has a length of at least about 0.8 meters, about 1.0 meter, about 1.2 meters, or about 1.4 meters. In embodiments, catheter body 114 has a length of no more than about 1.5 meters, about 1.0 meter, about 1.2 meters, or about 1.4 meters.

In embodiments, sleeve portion 276 of catheter 168 comprises a compliant material. Alternatively, sleeve portion 276 comprises a property selected from the group consisting of, flexible, plastic, and rigid.

In embodiments, catheter sleeve 276 has a wall thickness of at least about 0.2 millimeters, about 0.3 millimeters, or about 0.4 millimeters. In embodiments, catheter sleeve 276 has a wall thickness of no more than about 0.5 millimeters, about 0.4 millimeters, or about 0.3 millimeters.

Generally, collector 200, catheter 168, balloon 116, and all components thereof noted above, are manufactured using any one of a variety of biocompatible materials, for example, materials from the group including titanium, stainless steel, nitinol, shape memory metals, synthetic biostable polymer, a natural polymer, and an inorganic material.

Typical biostable polymers include a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, an aromatic polyester, a polyether (ether ketone), a polysulfone, a silicone rubber, a thermoset, or a polyester (ester imide) and/or combinations thereof.

Typical polymeric material includes a polyolefin, a polyurethane, a silicone, a polyester or a fluorinated polyolefin.

It is expected that during the life of this patent many relevant delivery systems will be developed and the scope of the AAPT collector 200 is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A catheter for disconnecting, collecting and removing an intraluminal mass from a luminal aspect of a blood vessel, comprising:
a) an elongate catheter body having a proximal portion and a distal portion;
b) an elongate tubular element extending proximally from an end of said proximal catheter body portion;
c) an intraluminal mass collector configured for collecting an intraluminal mass from a blood vessel, said collector having:
i) a body portion connected to said proximal portion of said catheter body; and
ii) a radially expandable portion extending in a proximal direction from said body portion and surrounding at least a portion of said elongate tubular element, said expandable portion having a reduced diameter configuration with a reduced cross sectional size and at least one expanded diameter configuration, each expanded diameter configuration having a respective expanded cross sectional size;
d) a disconnector configured for disconnecting an intraluminal mass from a luminal aspect of a blood vessel, said disconnector connected to a proximal portion of said elongate tubular element at a distance from said collector.

2. The apparatus according to claim 1, wherein said collector is configured to strain an intraluminal mass from the blood when in said expanded diameter configuration.

3. The apparatus according to claim 1, including a catheter sleeve slidably associated with at least a portion of said catheter body and configured to surround at least a portion of said collector in said reduced diameter configuration.

4. The apparatus according to claim 3, wherein said collector includes at least two substantially resilient rays extending from said collector body portion in a proximal direction towards an end of said radially expandable portion.

5. The apparatus according to claim 4 wherein said at least two rays comprise at least about 6 rays.

6. The apparatus according to claim 4, wherein said body portion of said intraluminal mass collector comprises a ring shaped component connected to said catheter body.

7. The apparatus according to claim 4, wherein proximal portions of each of said at least two rays are configured to resiliently flex outward to form at least one expanded cross sectional diameter.

8. The apparatus according to claim 7, wherein said expanded cross sectional diameter is at least about 3 centimeters.

9. The apparatus according to claim 7, wherein the extent of said outwards flexing is configured to be limited by the walls of a vessel in which said collector is deployed.

10. The apparatus according to claim 7, wherein each of said at least two rays is configured to resiliently flex outward to form said at least one expanded cross sectional diameter.

11. The apparatus according to claim 4, including a sheet material operatively associated with said at least two rays, said material forming a substantially conical shape pointing in a distal direction when said at least two rays are in a said expanded diameter configuration.

12. The apparatus according to claim 11, wherein said sheet material is selected from the group consisting of meshes and nets.

13. The apparatus according to claim 11, wherein said material includes openings having an area of at least about 0.25 mm².

14. The apparatus according to claim 11, wherein said material includes openings having an area of no more than about 4.0 mm².

15. The apparatus according to claim 11, wherein said material extends proximally beyond at least one of said at least two rays by at least about 1.0 millimeters.

16. The apparatus according to claim 11, wherein said material extends proximally beyond at least one of said at least two rays by no more than about 5.0 millimeters.

17. The apparatus according to claim 4, including at least one elongate flexible biasing element, having:
i) a first end attached to a first portion of said catheter sleeve;
ii) a second end attached to a second portion of said catheter sleeve; and
iii) a body between said first and second ends, said body being operatively associated with a proximal portion of each of said at least two rays.

18. The apparatus according to claim 17, wherein said at least one flexible biasing element is configured to bias at least one of said at least two rays from an expanded diameter configuration to a smaller diameter configuration.

19. The apparatus according to claim 17, wherein said at least one flexible biasing element has a diameter of at least 0.2 millimeters.

20. The apparatus according to claim 17, wherein said at least one flexible biasing element has a diameter of no more than about 0.8 millimeters.

21. The apparatus according to claim 17, further comprising a passage at a proximal portion of each of said at least two rays, through which said body of said at least one elongate flexible biasing element passes.

22. The apparatus according to claim 21, wherein said passage is formed from at least one of:
a bending of said proximal portion of said ray; and
a shaped component attached to said proximal portion of said ray.

23. The apparatus according to claim 1, wherein said catheter body includes a continuous aspiration channel from said distal portion and emerging into said collector body portion.

24. The apparatus according to claim 4, including a collector ray converger comprising a curved wall that slidingly substantially encircles a portion of said distal portion of said catheter body.

25. The apparatus according to claim 24, wherein said collector ray converger is additionally configured to encircle at least a portion of at least one of:
said collector; and
said catheter sleeve.

26. The apparatus according to claim 24, wherein said collector ray converger is configured to provide a radially inward force on at least one of said at least two rays.

27. The apparatus according to claim 24, wherein said collector ray converger is configured to reduce an intraluminal mass diameter, when said mass has been collected in said collector.

28. The apparatus according to claim 24, wherein said collector ray converger has a length of at least about 3 centimeters.

29. The apparatus according to claim 24, wherein said collector ray converger has a length of no more than about 7 centimeters.

30. The apparatus according to claim 24, wherein said collector ray converger wall has a thickness of at least about 0.3 millimeters.

31. The apparatus according to claim 24, wherein said collector ray converger wall has a thickness of no more than about 0.6 millimeters.

32. The apparatus according to claim 1, wherein said disconnector comprises a balloon configured to inflate by introduction of a fluid through an inflation channel running through said catheter body and said elongate tubular element.

33. The apparatus according to claim 32, wherein said balloon has a maximum inflation radius of at least about 2 centimeters.

34. The apparatus according to claim 1 wherein said distance from said disconnector to the proximal end of said collector in said reduced diameter configuration is at least about 5 centimeters.

35. The apparatus according to claim 1 wherein said distance from said disconnector to the proximal end of said collector in said reduced diameter configuration is no more than about 12 centimeters.

36. The apparatus according to claim 32, wherein said catheter body includes a substantially coaxial guide wire channel.

37. The apparatus according to claim 1, wherein said catheter body has an outside diameter of at least about 3.0 millimeters.

38. The apparatus according to claim 1, wherein said catheter body has an outside diameter of no more than about 5.5 millimeters.

39. The apparatus according to claim 3, wherein said catheter sleeve has a wall thickness of at least about 0.2 millimeters.

40. The apparatus according to claim 3, wherein said catheter sleeve has a wall thickness of no more than about 0.5 millimeters.

41. A method for collecting embolic debris within the vascular system, the method comprising:
opening a collector within a vascular system on a first side of embolic debris;
expanding an expandable lumen blocker on a second side of said embolic debris;
collecting said embolic debris within said collector;
closing said collector;
containing said embolic debris within said collector; and
removing said embolic debris from said vascular system.

42. The method according to claim 41, wherein said first side is proximal and said second side is distal to said embolic debris.

43. The method according to claim 41, wherein said first side is distal and said second side is proximal to said embolic debris.

44. The method according to claim 41, including treating a portion of a stenosed region using dilation.

45. The method according to claim 41, including treating a portion of a stenosed region using laser ablation.

46. The method according to claim 41, including treating a portion of a stenosed region by atherectomy.

47. The method according to claim 41, including aspirating said embolic debris from said collector.
